(19) United States
(12) Patent Application Publication
(10) Pub. No.: US 2011/0201996 A1
(43) Pub. Date: Aug. 18, 2011

(54) NASAL VENTILATION SYSTEM AND METHOD OF USING SAME

(76) Inventor: Patrick C. Melder, Marietta, GA (US)

(21) Appl. No.: 13/059,531

(22) PCT Filed: Aug. 27, 2009
(86) PCT No.: PCT/US09/55252

§ 371 Date: Mar. 9, 2011

(30) Foreign Application Priority Data
Aug. 27, 2008 (US) 61/092269

(51) Int. Cl. A61M 31/00

(52) U.S. Cl. 604/22; 604/48; 604/506

(57) ABSTRACT

An apparatus for creating an accessory maxillary ostium for purposes of ventilation, irrigation, or procedural work within the maxillary antrum. An introducer having a ventilation tube and grommet is provided to create and then provide access through the accessory maxillary ostium. An irrigation catheter may be advanced through the ventilation tube in order to irrigate or express the contents of the sinus cavities. A secondary irrigation catheter or a balloon catheter may be advanced through an outer irrigation catheter, deeply into the sinus cavity, in order to irrigate or express the contents of the sinus cavities. Moreover, the balloon catheter may alternatively or additionally be employed to for dilation of the natural ostium. Still further, a device for delivering drugs, fluids or the like, may be coupled to the ventilation tube for delivery of same.

### CROSS-REFERENCE TO RELATED APPLICATIONS

This application claims the benefit of U.S. Provisional Patent Application No. 61/092,269, filed Aug. 27, 2008, the entirety of which is hereby incorporated by reference.

### BACKGROUND OF THE INVENTION

1. Field of the Invention

The present invention relates, in general, to functional endoscopic techniques and, more particularly, to apparatus, systems and methods for ventilating the paranasal sinuses.

2. General Background of the Invention

There has been resurgence of interest among otolaryngologists in the morphological features of the lateral wall of nasal cavity with the advent of endonasal endoscopic sinus surgery. Functional endoscopic techniques, being minimally traumatic, have become increasingly popular in diagnostic and therapeutic aspects of nasal and sinus problems.

The area termed the “ostiomeatal complex” of the middle meatus has not only the primary maxillary ostia (“PMO”) opening in the hiatus semilunaris (“HS”) but also of-times, other “holes” or accessory maxillary ostium (AMO). It has been estimated that anywhere between 5-30% of the normal population has an AMO located in the maxillary fontanelle (anterior or posterior).

AMO is invariably solitary but occasionally multiple, either congenital or secondary to disease process. A possible mechanism of formation of accessory ostia is obstruction of the main ostium by maxillary sinusitis or due to anatomic and pathologic factors in the middle meatus resulting in the rupture of membranous areas known as fontanelle (certain regions in the middle meatus located below the uncinate process and above the inferior turbinate, covered by nasal mucous membrane medially and mucosa of maxillary sinus laterally with connective tissue sandwiched between the two).

In the past, a naso-antral window procedure was performed for purposes of ventilation and drainage of the maxillary sinus cavity. However, the naso-antral window procedure has the disadvantage of requiring one or more punctures through bony tissues.

Accordingly, it is an object of the present invention to provide apparatuses and methods for ventilation, irrigation, or procedural work within the maxillary antrum.

It is another object of the present invention to provide apparatuses and methods for the creation of an AMO to facilitate such ventilation, irrigation, or procedural work within the maxillary antrum.

It is another object of the present invention to provide an apparatus and method which may be used in connection with the sinuses, including the paranasal, ethmoid, and sphenoid sinuses.

It is yet another object of the present invention to provide apparatuses and methods for the creation of an AMO to facilitate such ventilation, irrigation, or procedural work within the maxillary antrum, without the requirement to puncture bony tissues, as in the prior naso-antral window procedure.

### BRIEF SUMMARY OF THE INVENTION

The present invention involves the creation of an AMO for purposes of ventilation, irrigation, or procedural work within the maxillary antrum. In particular, an introducer having a ventilation tube and grommet is provided to create and then provide access through the AMO. The irrigation catheter or balloon catheter may then be advanced through the ventilation tube in order to irrigate, or express the contents, of the sinus cavities. The balloon catheter may further be employed to dilate the natural ostium.

### BRIEF DESCRIPTION OF THE SEVERAL VIEWS OF THE DRAWINGS

FIG. 1 of the drawings is a sectional view of a human skull showing, in particular, the lateral nasal wall and the orientation of the introducer and ventilation tube;

FIG. 2A of the drawings is an enlarged view of a portion of the antrum, or maxillary sinus showing, in particular, the creation of an AMO using the introducer, and the placement of the ventilation tube through the AMO;

FIG. 2B of the drawings is an enlarged view of the antrum showing, in particular, an alternative embodiment of the ventilation tube;

FIG. 3 of the drawings is an enlarged view of the portion of the atrium showing, in particular, the obturator disposed through the introducer and the ventilation tube;

FIG. 4 of the drawings is an enlarged view of a portion of the lateral nasal wall showing, in particular, the ventilation tube in place after removal of the introducer;

FIG. 5 of the drawings is an enlarged view of a portion of the lateral nasal wall showing, in particular, the use of an irrigation catheter in association with the ventilation tube;

FIG. 6 of the drawings is an enlarged view of a portion of the lateral nasal wall showing, in particular, the use of a second, smaller diameter irrigation catheter within an outer irrigation catheter in association with the ventilation tube;

FIG. 7 of the drawings is an enlarged view of a portion of the lateral nasal wall showing, in particular, the second irrigation catheter extended deeply into the sinus cavity;

FIG. 8 of the drawings is an enlarged view of a portion of the lateral nasal wall showing, in particular, the use of a balloon catheter within the sinus cavity;

FIG. 9 of the drawings is an enlarged view of a portion of the lateral nasal wall showing, in particular, the placement of a balloon catheter within the natural ostium;

FIG. 10 of the drawings is an enlarged view of a portion of the lateral nasal wall showing, in particular, the expansion of the balloon catheter for dilation of the natural ostium;

FIG. 11 of the drawings shows a perspective view of another embodiment of the introducer of the present invention;

FIG. 12 of the drawings shows a perspective view of the introducer taken along lines 12-12 of FIG. 11.

FIG. 13A is a perspective view of the ventilation tube in accordance with the present invention.

FIG. 13B is a perspective view another embodiment of the ventilation tube in accordance with the present invention.

FIG. 14 is another embodiment of the catheter of the present invention, for delivering drugs and pharmaceuticals.

### DETAILED DESCRIPTION OF THE INVENTION

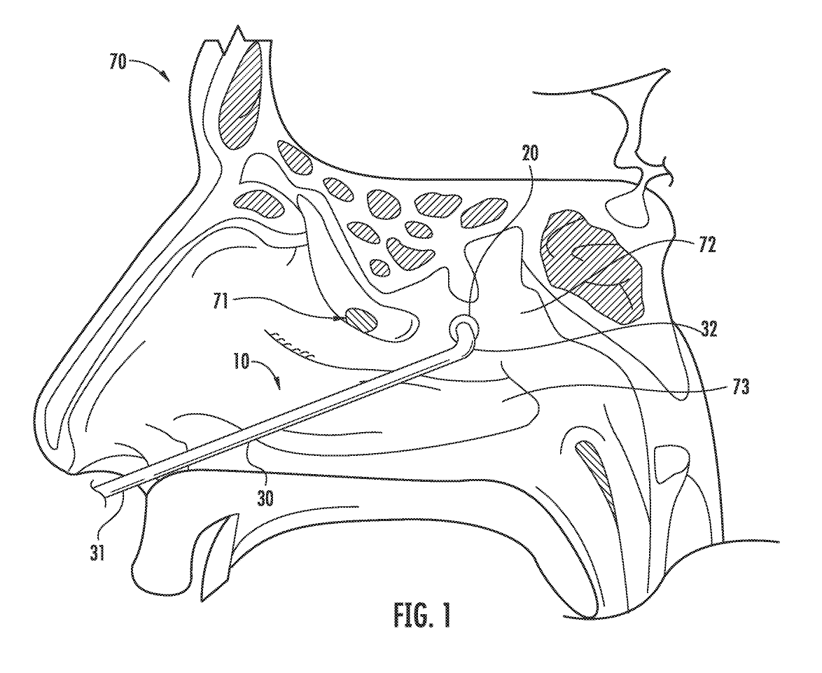

The present nasal ventilation system 10 is shown in FIG. 1 as comprising ventilation tube 20 and elongated introducer 30, suitable for creating an AMO within lateral nasal wall 70 (shown with the middle turbinate and uncinate removed to allow visualization of the natural ostium), proximate maxillary ostium 71, maxillary fontanelle 72, and inferior turbinate 73. A distal tip of introducer 30 is insertable through a central channel of ventilation tube 20, is releasably attachable to ventilation tube 20, and includes a sharp, removable cutting obturator at the distal tip.

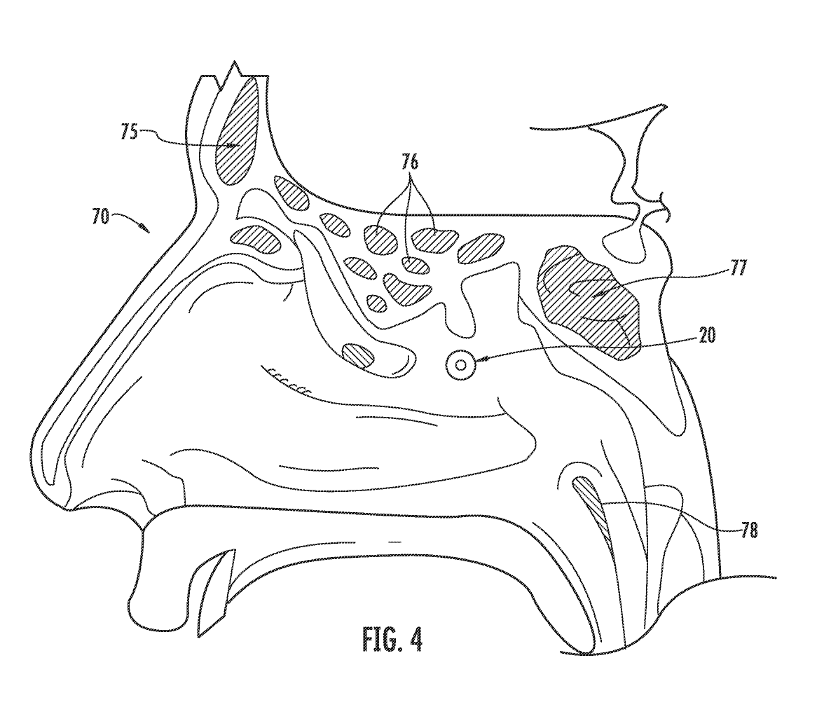

As shown in FIGS. 1 and 3, in operation, introducer 30 with ventilation tube 20 at its distal end are introduced into the middle meatus in the region of the fontanelle (anterior or posterior) under endoscopic assistance, facilitated by the obturator 100. Referring to FIG. 3, obturator 100 includes proximal flexible shaft 101 and sharp distal cutting tip 102. Once the membraneous fontanelle or other targeted area is identified, obturator 100 is advanced through introducer 30 and ventilation tube 20 until cutting tip 102 extends beyond ventilation tube 20, and cutting tip 102 and ventilation tube 20 are pushed through the fontanelle. Obturator 100 and introducer 30 are then removed. An endoscope is preferably employed to provide visualization during this procedure.

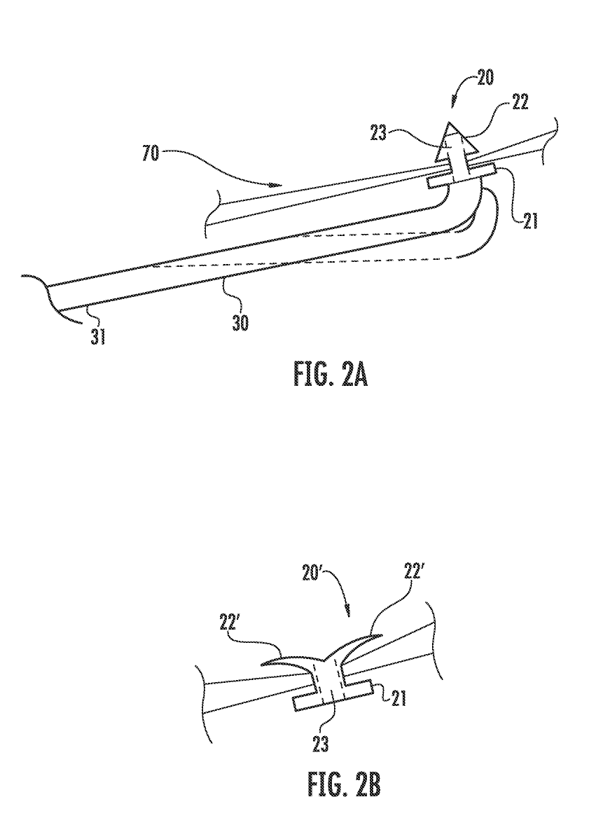

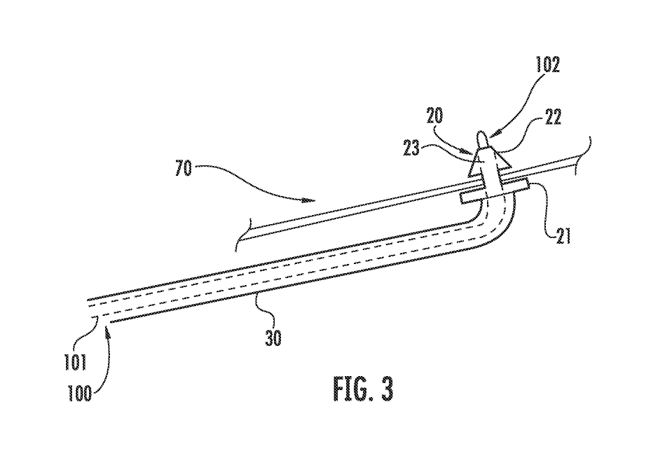

Ventilation tube 20 and introducer 30 are shown in further detail in FIG. 2A. Ventilation tube 20 include grommet-like member or proximal flange 21, retaining member or distal flange 22, and channel 23 extending through ventilation tube 20, terminating in opposing apertures extending through grommet-like member 21, retaining member 22 of ventilation tube 20. As shown in FIG. 2A, upon placement of ventilation tube 20, grommet-like member 21 and conical or frusto-conical retaining member 22 are disposed on opposing sides of the AMO extending through lateral nasal wall 70, and serve to maintain ventilation tube 20 in place. As indicated by the phantom lines of FIG. 2A, introducer 30 is preferably constructed of a relatively flexible material to permit the introducer 30 to be readily advanced through the paranasal sinus passages to the desired site of the AMO.

Ventilation tube 20 may be constructed of a variety of plastic-like materials commonly used in medical devices, including materials commonly employed in middle ear ventilation tubes. Moreover, ventilation tube 20 may be constructed of a drug eluting material, and may include compounds such as steroids or minerals/elements to decrease viral contamination, inflammatory reactions, and bacterial colonization. Furthermore, ventilation tube 20 may be constructed of a resorbable material, similar to those used in bio-absorbable sutures, and capable of dissolving in situ over time.

An alternative construction of the ventilation tube, namely ventilation tube 20′, is shown in FIG. 2B. In this alternative embodiment, the retaining member comprises expandable feet 22′ serving, in cooperation with grommet-like member 21, to retain ventilation tube 20 in place though the membranous fontanelle.

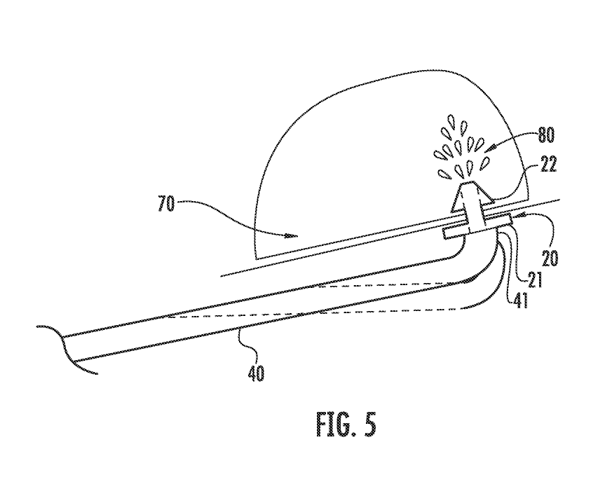

As shown in FIG. 4, once ventilation tube 20 is placed and seated through lateral nasal wall 70 proximate frontal sinus 75, ehtnoid sinus 76, sphenoid sinus 78 and Eustachian tube 78, introducer 30 and its associated obturator are separated from ventilation tube 20 and removed. Next, as shown in FIG. 5, irrigation catheter 40 may be placed, by inserting distal tip 41 of irrigation catheter 40 into, or entirely through, channel 23 of ventilation tube 20. Irrigation fluid 80 may then be injected into irrigation catheter 40 and thus introduced into the desired treatment area to flush the contents of the sinus cavity. The contents of the sinus cavity can then be pushed through the principal or main maxillary ostium to un-block the natural ostium. As indicated by the phantom lines of FIG. 5, irrigation catheter 40 is preferably constructed of a relatively flexible material to permit distal tip 41 of irrigation catheter to be advanced into the paranasal sinuses and placed through ventilation tube 20.

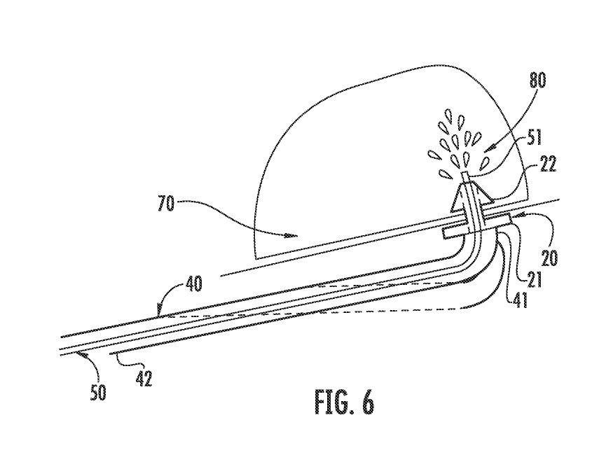

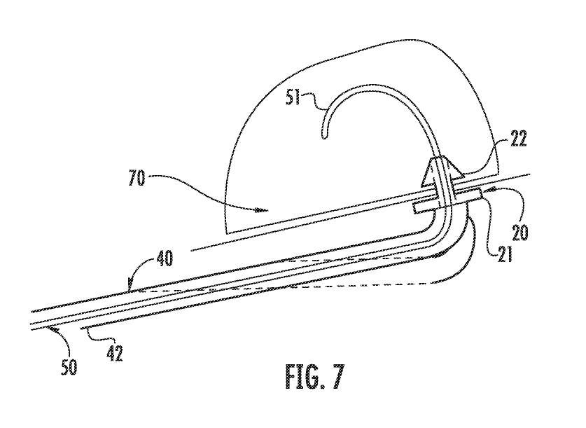

As shown in FIG. 6, a separate, smaller diameter secondary irrigation catheter 50 may be used in conjunction with irrigation catheter 40. Distal tip 51 of secondary catheter 50 is inserted through an opening at proximal end 42 of irrigation catheter 40, and is advanced beyond distal tip 41 of irrigation catheter 40 and into the targeted sinus cavity. Irrigation fluid 80 is then injected into secondary irrigation catheter 50 and is thus introduced to the desired treatment area to flush the contents of the sinus cavity. Moreover, and as shown in FIG. 7, distal tip 51 of secondary catheter 50 may be inserted more deeply into the targeted sinus cavity to deeply instill liquids within the antrum, by further advancing secondary catheter 50 through irrigation catheter 40.

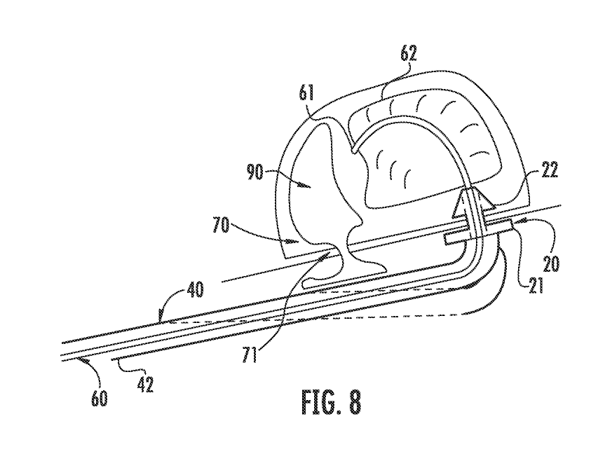

Alternatively, and as shown in FIG. 8, balloon catheter 60 may be employed in place of secondary irrigation catheter 50. Balloon catheter 60 includes distal tip 61, and expansile member 62 surrounding a distal portion of the main lumen of the balloon catheter 60. As shown in FIG. 8, distal tip 61 may be placed through irrigation catheter 40 and advanced deeply into the targeted sinus cavity. Expansile member 62 is then inflated. This, in turn, causes the contents of the sinus cavity, such as mucous or purulent debris 90, to be expressed through the natural PMO 71.

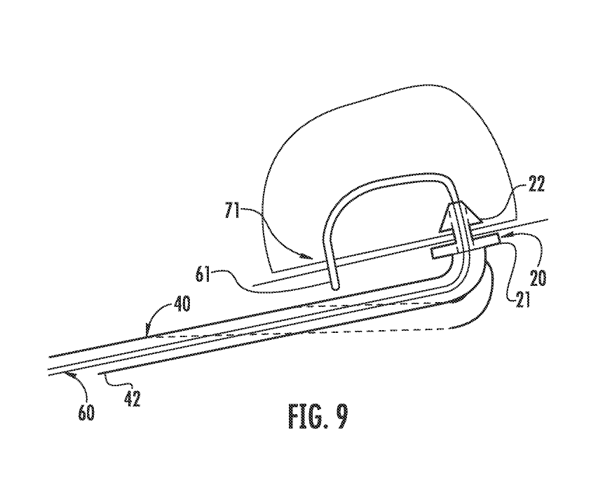

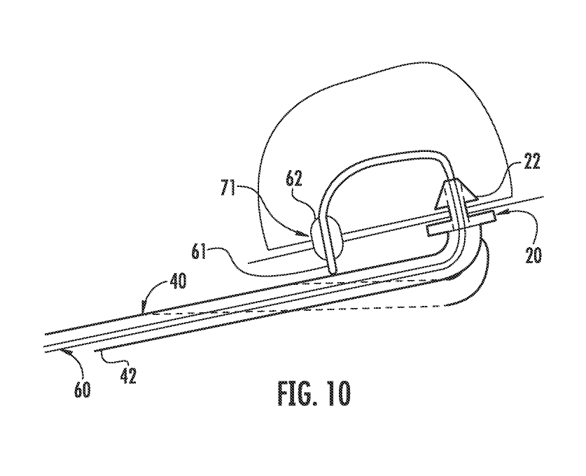

As shown in FIG. 9, distal tip 61 of balloon catheter 60 may alternative or additionally be advanced through irrigation catheter 40 and the sinus cavity to extend through the natural PMO 71. Next, as shown in FIG. 10, expansile member 62 is inflated, in order to dilate the natural PMO 71.

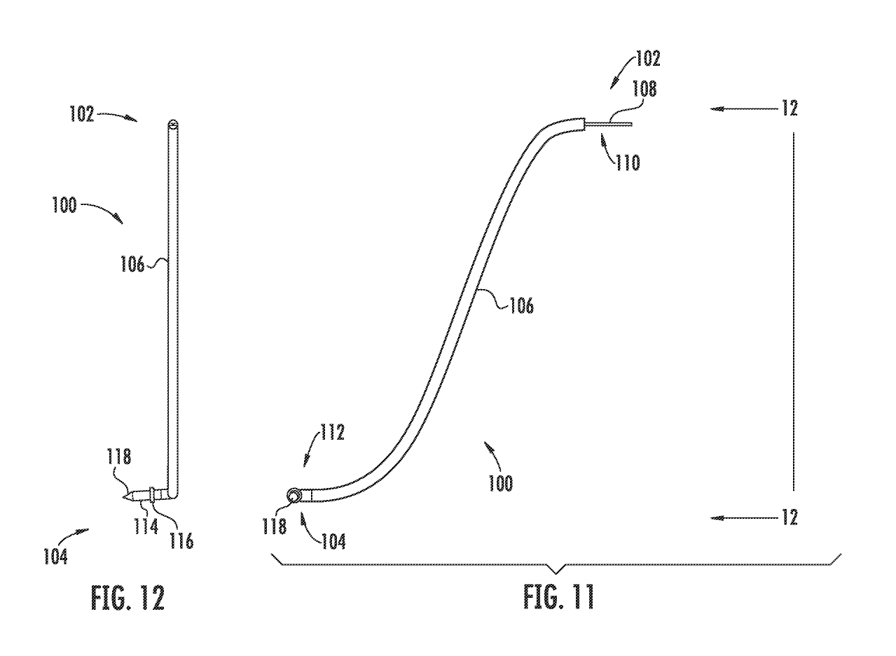

FIG. 11 is a view of another embodiment of an elongated introducer 100. The introducer 100 includes a proximal end 102 and a distal end 104. A tube or shaft 106 extends between the proximal end 102 and distal end 104. A rod 108 extends within the tube 106. The rod 108 includes a proximal end 110 extending out of tube 106 and a distal end (not shown) located at the distal end 104 of the introducer 100. An obturator 112 is located at the distal end 104. In one embodiment, the obturator 112 is secured to the distal end of the rod 108. FIG. 12 is a view of the elongated introducer 100 taken along line 12-12 of FIG. 11. FIG. 12 shows the obturator 112 includes a shaft 114, a flange 116 and a sharp, distal cutting tip 118. The shaft 114 may be cylindrical or conical shaped, for example. The elongated introducer 100 may be coupled to a myringotomy apparatus or handle (not shown), such as will be understood in the art. Such an apparatus typically includes a mechanism to secure the tube 106 in a stationary manner with respect to the apparatus. The rod 108 may be coupled to a trigger mechanism for slidingly activating and controlling the rod 108 with respect to the tube 106. Thus, activation of the trigger of the apparatus controls movement of the obturator 112. The tube 106 of the introducer 100 may be made of a flexible or malleable material to permit the introducer 100 to be readily advanced through the paranasal sinus passages to the desired site of the AMO.

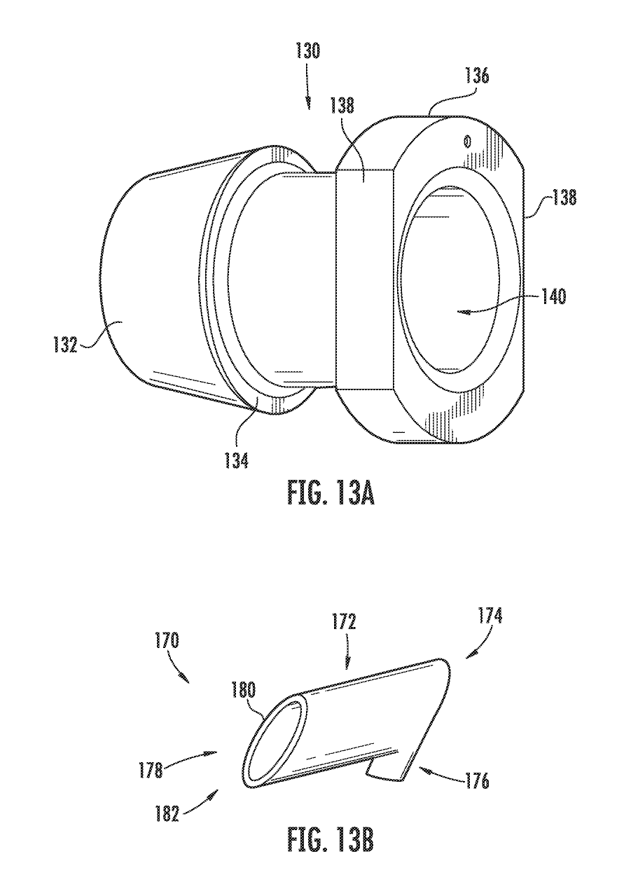

FIG. 13A shows another embodiment of the ventilation tube 130. The ventilation tube 130 includes the conical or frusto-conical retaining member 132, a shoulder 134, grommet-like member 136 having flat side edges 138, and a central channel or bore 140 extending through the ventilation tube 130.

As understood from the above description, the obturator 112 receives the ventilation tube 130. In particular, the shaft 114 is designed to be received by the central channel 140. The shaft 114 and channel 140 may be designed to provide a releasable locking engagement, such as an interference press fit or a snap-fit engagement. The shaft 114 may be cylindrical or conical, for example, with the central channel 140 similarly shaped for a mating engagement. With the obturator 112 inserted into the ventilation tube 130, the flange 116 and grommet-like member 136 limit the depth in which the obturator 11 is inserted within the central channel 140. With the flange 116 engaging the grommet-like member 136, the sharp distal cutting tip 118 projects beyond the conical retaining member 132. Thus, the combined introducer 100 and ventilation tube 130 are adaptable for piercing the tissue and forcing the ventilation tube 130 in place, with the shoulder on one side of the wall and the grommet-like member on the other side of the wall.

In another embodiment, the ventilation tube may take other forms such as omitting the distal flange. In FIG. 13B, another embodiment of the ventilation tube is shown. In particular, ventilation tube 170 shows a neck portion or sleeve 172 having a proximal end 174 with a proximal flange 176. As an example, the flange may be tab shaped as shown in FIG. 13B or annular shaped. The distal end 178 includes a beveled edge 180 which provides a sharp cutting tip 182. The ventilation tube 170 shown in FIG. 13B is more readily inserted by use of forceps (not shown). For example, the forceps may grip the proximal flange or tab 176. The forceps will be used to guide the tube in place, urging the sharp cutting tip 182 through the sinus wall, and wherein the tab will also limit the insertion depth of the ventilation tube. It will be appreciated that an obturator is not required for inserting the tube 170 into the wall.

In addition, it will be appreciated that the forceps may be used instead of the introducer described above. However, when the term “introducer” is used herein, it is intended to include forceps.

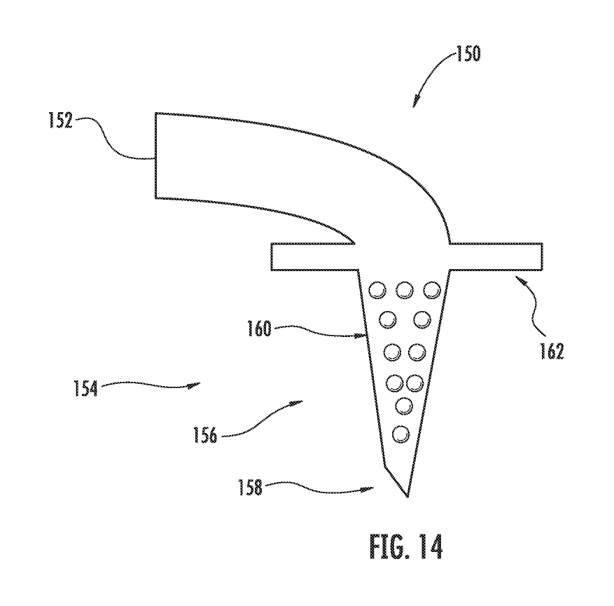

FIG. 14 shows another embodiment of a catheter used utilized for delivering a pharmaceutical drug, fluid or the like to the sinus cavity. The catheter 150 is a graphical representation. The catheter 150 includes a hollow shaft 152. The shaft 152 may be of a malleable material or flexible material to permit the catheter to be readily advanced through the sinus passages to the desired site of the AMO, and in particular to the location of the ventilation tube. It will be appreciated that the shaft 152 will be proportionally longer than that shown in FIG. 14. The distal end 154 of the catheter 150 includes an irrigation tip 156. The irrigation tip 156 may be generally conical in shape. The conical shape more readily accommodates insertion of the tip 156 into the ventilation tube 130. The irrigation tip 156 may be hollow to provide for fluid and drug flow from the shaft. The irrigation tip 156 may include a delivery opening 158 at the distal end 154. In addition, the wall of the irrigation tip 156 may include micro pore perforations 160. The delivery openings 158 and micro pore perforations 160 provide delivery of the drug or fluid. A flange 162 is provided to limit the insertion depth of the catheter 150. The flange 162 will engage the grommet-like member to limit the insertion of the catheter.

It will be appreciated that the ventilation tube of the present invention may be used to deliver topical fluids, drugs, anti-inflammatory medications, such as steroids, gene treatments, etc., drug delivery substances, and drug impregnated coils, and beads through simple insertion or via powered pulsation. For example, a device for delivering such items, such as the catheter 150 for example, may be guided toward the inserted ventilation tube, whereupon the desired item may be delivered directly to the sinus, or in a manner consistent with the description herein. In one embodiment, the delivery device may be adapted for insertion into the first catheter, to assist in guiding the delivery device to the ventilation tube.

What is claimed is:

1. A nasal ventilation apparatus comprising:
a ventilation tube; and
an introducer releasably attachable to the ventilation tube.

2. The invention according to claim 1, further comprising a cutting obturator associated with the introducer, or wherein the ventilation tube includes a sharp cutting distal tip.

3. The invention according to claim 1, wherein the ventilation tube is configured to be disposed through the membraneous fontanelle of the lateral nasal wall.

4. The invention according to claim 3, wherein the ventilation tube is configured to be secured in place proximate the membraneous fontanelle of the lateral nasal wall.

5. The invention according to claim 4, wherein the ventilation tube includes at least one retaining member securing the ventilation tube in place proximate the membraneous fontanelle of the lateral nasal wall.

6. The invention according to claim 1, wherein the ventilation tube further comprises a proximal flange and a distal flange, with a neck portion therebetween, and a channel extending through at least a portion of the ventilation tube, the distal flange having a conical shape with the diameter decreasing in a distal direction.

7. The invention according to claim 1, wherein the ventilation tube further comprises a neck portion extending from a proximal flange or tab, and a channel extending through at least a portion of the ventilation tube, with a sharp cutting distal tip.

8. The invention according to claim 1, further comprising a first irrigation catheter, the first irrigation catheter being at least one of attachable to and extendable through at least a portion of the ventilation tube.

9. The invention according to claim 8, further comprising a second irrigation catheter extendable through at least a portion of the first irrigation catheter or a balloon catheter extendable through at least a portion of the first irrigation catheter.

10. The invention according to claim 1, further comprising a delivery device or catheter configured to be coupled to the ventilation tube for delivery of topical fluids, drugs, anti-inflammatory medications, drug delivery substances, drug impregnated coils, foams, or beads, wherein optionally a distal end of the device may include a proximal flange and an irrigation tip extending from the proximal flange, the irrigation tip having a conical shaped wall with a distal tip, with an opening at the distal tip or perforations in the conical shaped wall.

11. The invention according to claim 1, wherein the introducer includes a tube having a proximal end and a distal end, with a shaft extending within the tube, the shaft includes a proximal end adaptable for coupling to an actuator mechanism, and a distal end having an obturator extending from the distal end of the tube, the obturator includes a flange with a cylindrical shaft extending distally from the flange, and a sharp distal cutting tip, wherein the actuator mechanism may cause longitudinal movement of the shaft and correspondingly the obtuator.

12. The invention of claim 10, wherein the introducer tube is made of a flexible material, malleable material or a metal or similar material, including nitinol.

13. A method of ventilating the sinus, comprising the steps of:
guiding a ventilation tube and a sharp cutting distal tip toward the wall of a sinus cavity using a first tool;
puncturing the wall with the sharp cutting distal tip;
pressing the ventilation tube partially through the wall, until a proximal flange is adjacent the sinus wall;
retracting the first tool with the ventilation tube in place with the proximal flange adjacent the side of the sinus cavity wall; and
guiding a further tool toward the ventilation tube and operating the tool.

14. The method of claim 13, wherein the step of guiding a further tool includes guiding a first irrigation catheter and coupling the irrigation catheter to the ventilation tube, and injecting irrigation fluid into the irrigation catheter and thus into the desired treatment area.

15. The method of claim 14, wherein a further irrigation catheter is inserted into the first irrigation catheter and injecting irrigation fluid into the irrigation catheter and thus into the desired treatment area, wherein the further irrigation catheter may be extended beyond a distal tip of the first irrigation catheter.

16. The method of claim 14, further comprising the step of inserting a balloon catheter into the first irrigation catheter, wherein the balloon is expanded in the nasal cavity or the natural PMO.

17. The method of claim 13, wherein the step of guiding a further tool includes guiding a catheter or similar device towards the ventilation tube, wherein the further tool includes an irrigation tip, the irrigation tip having a delivery opening at the distal end or the wall of the irrigation tip may include micro pore perforations, the delivery openings and micro pore perforations provide delivery of the drug or fluid, a flange is provided to limit the insertion depth of the catheter, the flange will engage the grommet-like member to limit the insertion of the catheter.

18. The method of claim 13, wherein the step of guiding a further tool includes guiding a device or catheter toward the ventilation tube and delivering topical fluids, drugs, anti-inflammatory medications, drug delivery substances, drug impregnated coils, foams, or beads via insertion or via powered pulsation.

19. The method of claim 13, wherein the step of guiding includes guiding an introducer releasably attachable to the ventilation tube.

20. A ventilation sinus kit comprising a ventilation tube; and an introducer adaptable for releasable attachment to the ventilation tube, and a further tool such as a first irrigation catheter, a further irrigation catheter adapted for insertion into the first irrigation catheter, a balloon catheter adapted for insertion into the first irrigation catheter, forceps, or a delivery device.
